(19) United States
(12) Patent Application Publication
(10) Pub. No.: US 2009/0131809 A1
(43) Pub. Date: May 21, 2009

(54) Respiration sensor

(76) Inventors: James Huang, HsinChu (TW); Jason Liao, HsinChu (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/292,350

(22) Filed: Nov. 18, 2008

(30) Foreign Application Priority Data
Nov. 20, 2007 (TW) 96143835

(51) Int. Cl. A61B 5/08

(52) U.S. Cl. 600/529; 600/534

(57) ABSTRACT

The present invention discloses a respiration sensor, which is attached to a testee and comprises a triaxial acceleration sensing unit, a microprocessor, and at least one alarm unit. The triaxial acceleration sensing unit detects the respiration-related movements of a testee and output a detection signal. The microprocessor connects with the triaxial acceleration sensing unit, receives the detection signal and transforms the detection signal into a respiration signal to determine the respiration state of the testee. When the respiration signal is lower than a standard, the microprocessor triggers the alarm unit to give out an alarm to alert the persons nearby.

### BACKGROUND OF THE INVENTION

1. Field of the Invention

The present invention relates to a respiration sensor, particularly to sensor used to detect abnormal respiration.

2. Description of the Related Art

People spend about one third to one fourth of their lifetime on sleep. However, some persons have respiration disturbance or apnea during sleeping, which may bring about suffocation, death or brain anoxia. Respiration disturbance or apnea may result from a respiratory tract clogging, a deficient cardiopulmonary function, or a poor sleeping environment.

There have been some products or patents for monitoring respiration or heartbeat of infants, babies, elders, or patients of respiration disturbance and cardiopulmonary function deficiency. As shown in FIG. 1, a conventional infant respiration monitor 10 includes a sensing sheet 12 and a host 14 coupled to the sensing sheet 12. An infant lies on the sensing sheet 12; the sensing sheet 12 senses the movements of the infant and then transmits signals to the host 14. However, the respiration monitor 10 is very expensive. Besides, it is apt to have a false output when the infant is heavily clothed.

A Taiwan patent of application No. 090120282 disclosed a “Sleep Apnea Detection System and Method”. The device includes a microphone and a controller coupled to the microphone. The microphone detects the sounds of respiration and transmits signals to the controller. The controller can recognize at least one respiration mode of sleep apnea. The microphone and controller are attached to a detachable collar, and then the detachable collar is worn the neck of a testee. Naturally, wearing the collar on the neck will make the testee uncomfortable.

Accordingly, the present invention proposes a novel respiration sensor to solve the conventional problems.

### SUMMARY OF THE INVENTION

One objective of the present invention is to provide a respiration sensor, which uses a triaxial acceleration sensing unit (g-sensor) to detect the respiration-related movements, identifies whether respiration is normal, and then outputs an alarm when respiration is abnormal.

Another objective of the present invention is to provide a wireless respiration sensor, whereby a caregiver can learn the respiration state of a testee anytime via a far-end receiver device although the caregiver is not beside the testee.

The present invention proposes a respiration sensor, which is attached to a testee and comprises a triaxial acceleration sensing unit, an alarm unit, a microprocessor and a power supply unit. The triaxial acceleration sensing unit detects the respiration-related movements of a testee and outputs detection signals. The microprocessor receives the detection signals and transforms the detection signals into a respiration signal to determine the respiration state of the testee. When the respiration signal is lower than a standard, the microprocessor triggers the alarm unit to give out an alarm to alert the persons nearby.

The present invention also proposes a respiration sensor, which is attached to a testee and comprises a triaxial acceleration sensing unit, an alarm unit, a microprocessor, a power supply unit and a wireless transmitter. The microprocessor is connected with the triaxial acceleration sensing unit, alarm unit and wireless transmitter. The triaxial acceleration sensing unit detects the respiration-related movements of a testee and outputs detection signals. The microprocessor receives the detection signals and transforms the detection signals into a respiration signal to determine the respiration state of the testee. When the respiration signal is lower than a standard, the microprocessor triggers the alarm unit to give out an alarm to alert the persons nearby. The wireless transmitter also constantly transmits the respiration signal to a far-end receiver device, whereby a far-end monitor can learn the respiration state of the testee anytime. When the respiration signal is lower than a standard, the receiver device also gives out an alarm to alert the far-end monitor.

The embodiments will be described in detail to enable the person skilled in the art to easily understand the present invention.

The foregoing schematic description and following detailed description are to demonstrate the present invention and support the claims of the present invention.

### BRIEF DESCRIPTION OF THE DRAWINGS

FIG. 1 is a diagram schematically showing a conventional respiration monitor;

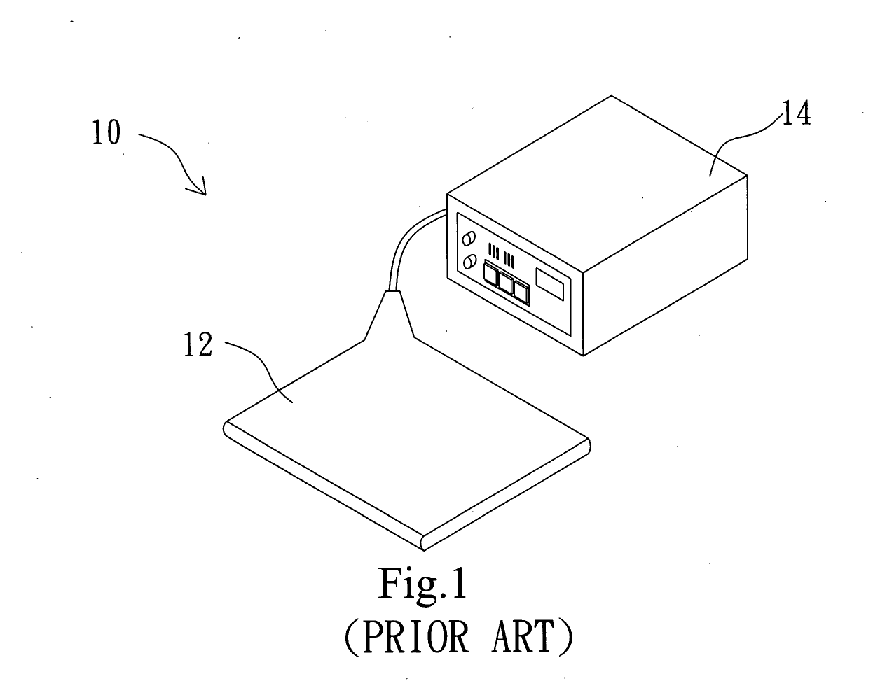

FIG. 2 is a diagram schematically showing the appearance of a respiration sensor according to the present invention;

FIG. 3 is a diagram schematically showing the circuit structure of a respiration sensor according to the present invention;

FIG. 3 is a diagram schematically showing the application of the present invention;

FIG. 5 is a diagram schematically showing the appearance of another respiration sensor according to the present invention; and

FIG. 6 is a diagram schematically showing the circuit structure of another respiration sensor according to the present invention.

### DETAILED DESCRIPTION OF THE INVENTION

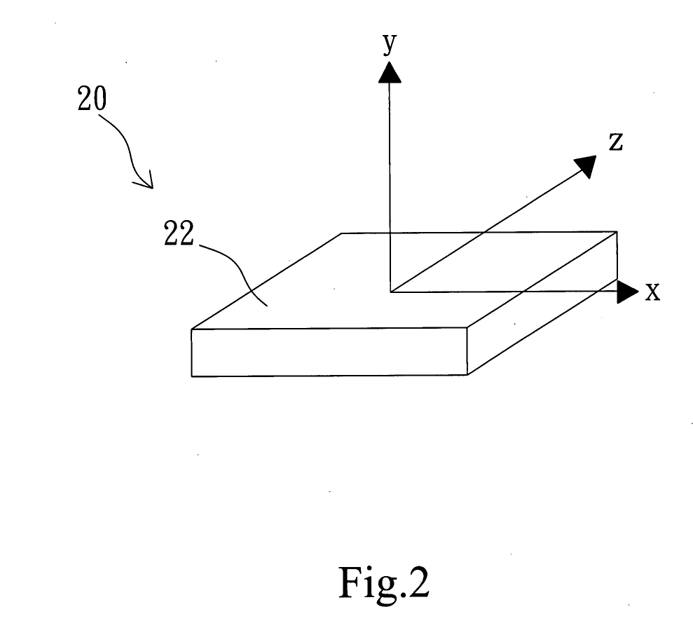

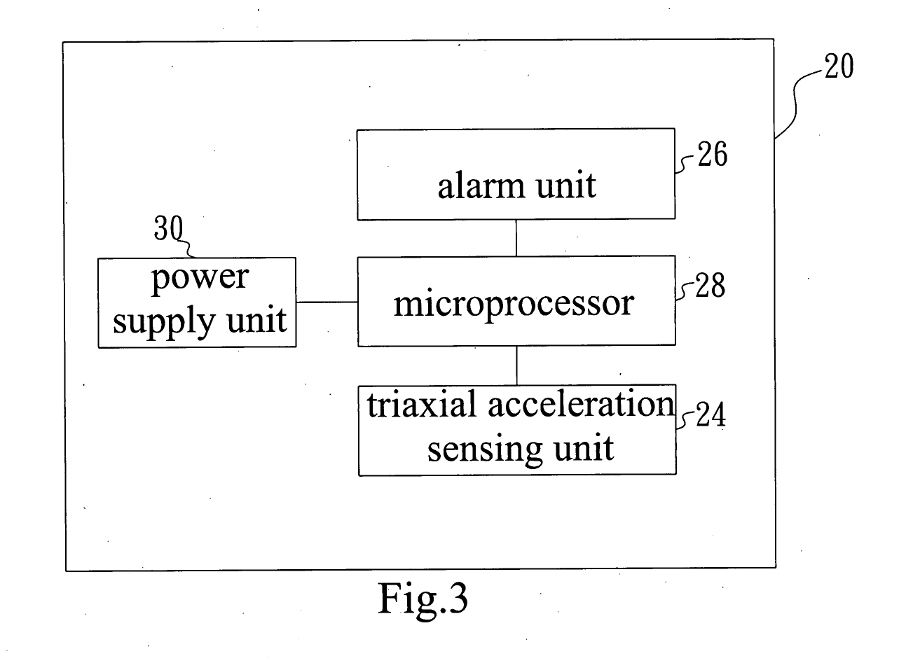

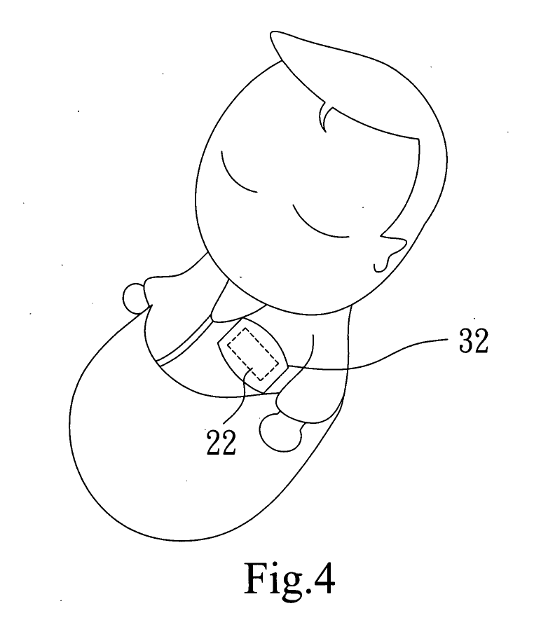

Refer to FIG. 2 and FIG. 3 diagrams respectively schematically showing the appearance and the circuit structure of a respiration sensor according to the present invention. The respiration sensor 20 of the present invention comprises a body 22, a triaxial acceleration sensing unit (g-sensor) 24, an alarm unit 26, a microprocessor 28, and a power supply unit 30. The triaxial acceleration sensing unit (g-sensor) 24, alarm unit 26, microprocessor 28, and power supply unit 30 are all arranged inside the body 22. The microprocessor 28 is connected to the triaxial acceleration sensing unit 24 and the alarm unit 26 and controls the operations of the components. The power supply unit 30 provides power for the components. The alarm unit 26 is an indicator, such as an LED light able to light up or flash. Alternatively, the alarm unit 26 is a buzzer giving out alerting sounds.

When detecting the respiration of a testee, the respiration sensor 20 is placed on the chest or abdomen of the testee. Below, the present invention is exemplified with the case that the respiration sensor 20 is placed on the chest. Refer to FIG. 4. The body 22 is stuck to the chest of a testee by an adhesive tape 32. Then, the triaxial acceleration sensing unit 24 detects the movements of the chest and outputs a detection signal to the microprocessor 28. The detection signal is a voltage signal. The microprocessor 28 receives the detection signal and transforms the detection signal into a respiration signal to determine the respiration state of the testee. When the respiration signal is lower than a standard, the microprocessor 28 triggers the alarm unit 26 to give out an alarm, such as lights or sounds, to alert the persons nearby. The body 22 of the respiration sensor 20 may have a clip element (not shown in the drawings) able to clip to the clothes of the testee, whereby the body 22 can be fixed to the chest of the testee.

With the adhesive tape or the clip element, the respiration sensor 20 can also be fixed to the abdomen of the testee to detect the movements of the abdomen during respiration and determine the respiration state of the testee. The case that the respiration sensor 20 is placed on the abdomen is similar to the case on the chest and will not repeat in detail herein.

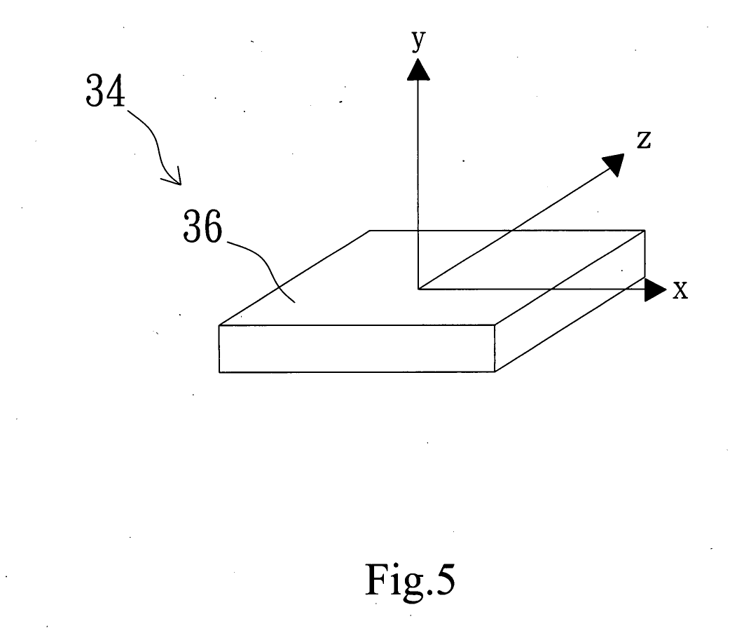

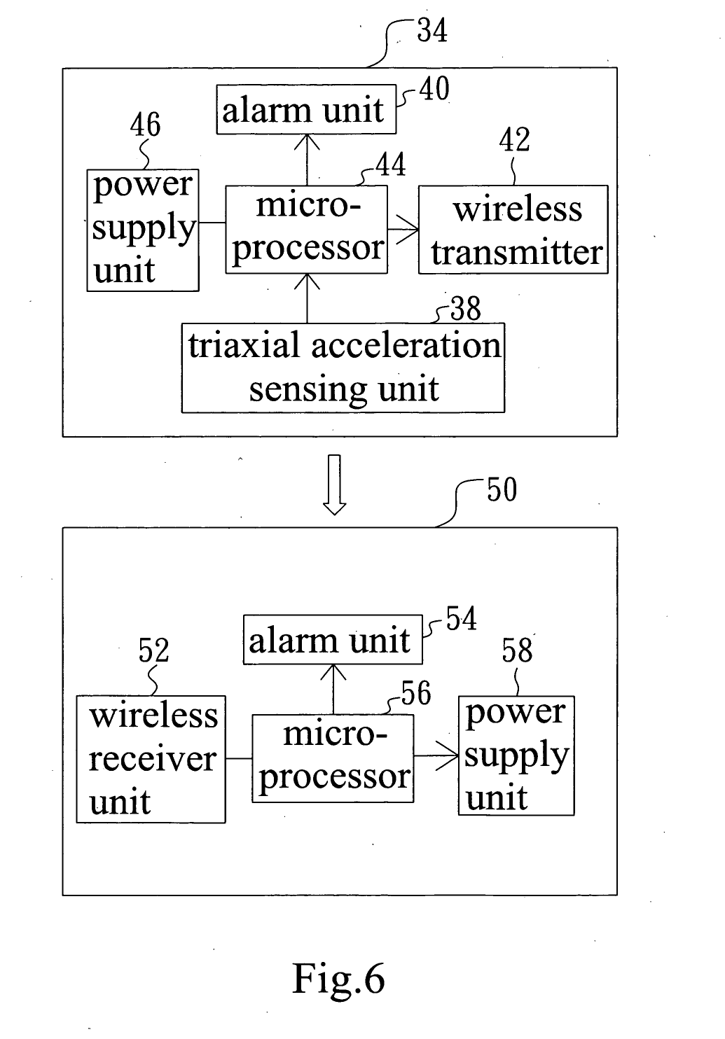

Refer to FIG. 5 and FIG. 6. FIG. 5 is a diagram schematically showing the appearance of another respiration sensor according to the present invention. FIG. 6 is a diagram schematically showing the circuit structures of another respiration sensor and a receiver device according to the present invention. The respiration sensor 34 of the present invention is a wireless respiration sensor. The respiration sensor 34 can operate singly or collaborate with a far-end receiver device 50. The respiration sensor 34 comprises a body 36, a triaxial acceleration sensing unit (g-sensor) 38, an alarm unit 40, a wireless transmitter 42, a microprocessor 44, and a power supply unit 46. The triaxial acceleration sensing unit 38, alarm unit 40, wireless transmitter 42, microprocessor 44, and power supply unit 46 are all arranged inside the body 36. The microprocessor 44 is connected to the triaxial acceleration sensing unit 38, alarm unit 40 and wireless transmitter 42 and controls the operations of the components. The power supply unit 46 provides power for the components. The alarm unit 40 is an indicator, such as an LED light able to light up or flash. Alternatively, the alarm unit 40 is a buzzer giving out alerting sounds. The far-end receiver device 50 comprises a wireless receiver unit 52, an alarm unit 54, a microprocessor 56, and a power supply unit 58. The microprocessor 56 is connected to the wireless receiver unit 52 and the alarm unit 54 and controls the operations of the components. The alarm unit 54 is an indicator, such as an LED light able to light up or flash. Alternatively, the alarm unit 40 is a buzzer or a vibrator.

After the body 36 of the respiration sensor 34 is fixed to the chest of a testee with an adhesive tape or a clip element (not shown in the drawing), the triaxial acceleration sensing unit 38 begins to detect the movements of the chest of the testee and outputs a detection signal to the microprocessor 44. The detection signal is a voltage signal. The microprocessor 44 receives the detection signal and transforms the detection signal into a respiration signal to determine the respiration state of the testee. When the respiration signal is lower than a standard, the microprocessor 44 triggers the alarm unit 40 to give out an alarm, such as lights or sounds, to alert the persons nearby. At the same time, the microprocessor 44 controls the wireless transmitter 42 to transmit the respiration signal to the far-end receiver device 50. The wireless receiver unit 52 of the far-end receiver device 50 receives the respiration signal and transmits the respiration signal to the microprocessor 56. When the respiration signal is lower than a standard, the microprocessor 56 triggers the alarm unit 54 to give out an alarm, such as lights, sounds, or vibrations, to alert the far-end monitor. Therefore, the cooperation of the wireless respiration sensor 34 and the wireless far-end receiver device 50 can reduce the burden of the family or caregivers. Although the family or caregivers are not beside the testee, they can still learn the respiration state of the testee.

With the adhesive tape or the clip element, the respiration sensor 34 can also be fixed to the abdomen of the testee to detect the movements of the abdomen during respiration and determine the respiration state of the testee. The case that the respiration sensor 34 is placed on the abdomen is similar to the case on the chest and will not repeat in detail herein.

In conclusion, the present invention proposes a respiration sensor, which detects the movements of the testee's body to determine whether the respiration state of the testee is normal. The present invention is easy-to-operate and has a small size. Therefore, the present invention will not discomfort the testee. Further, the present invention is inexpensive and unlikely to output a false result.

The embodiments described above are to exemplify the present invention to enable the persons skilled in the art to understand, make, and use the present invention. However, it is not intended to limit the scope of the present invention. Therefore, any equivalent modification or variation according to the spirit of the present invention is to be also included within the scope of the present invention.

What is claimed is:

1. A respiration sensor, attached to a body region of a testee and comprising
a triaxial acceleration sensing unit detecting body movements of said testee and outputting a detection signal;
a first microprocessor coupled to said triaxial acceleration sensing unit, receiving said detection signal, and transforming said detection signal into a respiration signal to determine a respiration state of said testee;
at least one first alarm unit coupled to said first microprocessor and triggered by said first microprocessor to give out an alarm signal when said respiration signal is lower than a standard;
a first power supply unit providing power; and
a body receiving components mentioned above.

2. The respiration sensor according to claim 1, wherein said detection signal is a voltage signal.

3. The respiration sensor according to claim 1, wherein said first alarm unit is a buzzer.

4. The respiration sensor according to claim 1, wherein said first alarm unit is an indicator.

5. The respiration sensor according to claim 1, wherein said body is stuck to said testee with an adhesive tape.

6. The respiration sensor according to claim 1, wherein said body is fixed to said testee with a clip element.

7. The respiration sensor according to claim 1 further comprising a wireless transmitter arranged inside said body, coupled to said first microprocessor, and transmitting said respiration signal to a far-end receiver device to inform of a respiration state of said testee.

8. The respiration sensor according to claim 7, wherein said far-end receiver device further comprises
a wireless receiver unit receiving said respiration signal transmitted by said wireless transmitter;
a second microprocessor coupled to said wireless receiver unit and receiving said respiration signal;
at least one second alarm unit coupled to second microprocessor and triggered by said second microprocessor to give out an alarm signal when said respiration signal is lower than a standard; and
a second power supply unit providing power.

9. The respiration sensor according to claim 8, wherein said second alarm unit is an indicator.

10. The respiration sensor according to claim 8, wherein said second alarm unit is a buzzer.

11. The respiration sensor according to claim 8, wherein said second alarm unit is a vibrator.

12. The respiration sensor according to claim 8, wherein said body region is a chest region or an abdominal region.
